(19) United States
(12) Patent Application Publication
(10) Pub. No.: US 2019/0218284 A1
(43) Pub. Date: Jul. 18, 2019

(54) METHODS OF TREATING EOSINOPHILIC GASTROINTESTINAL DISEASES

(71) Applicant: THE REGENTS OF THE UNIVERSITY OF COLORADO a body corporate, Denver, CO (US)

(72) Inventors: Glenn FURUTA, Centennial, CO (US); Joanne MASTERSON, Denver, CO (US)

(21) Appl. No.: 16/327,045

(22) PCT Filed: Aug. 31, 2017
(86) PCT No.: PCT/US17/49515

§ 371 Date: Feb. 21, 2019

Related U.S. Application Data
(60) Provisional application No. 62/382,653, filed on Sep. 1, 2016.

(51) Int. Cl. C07K 16/24; A61P 1/00

(52) U.S. Cl. CPC C07K 16/243; A61K 2039/505; C07K 2317/76; A61P 1/00

(57) ABSTRACT

Methods of treating or preventing Eosinophilic Gastrointestinal Diseases (EGIDs), including eosinophilic esophagitis (EOE) by administering anti-GM-CSF antibodies.

### CROSS REFERENCE TO RELATED APPLICATION

This application claims the benefit of priority under 35 U.S.C. § 119(e) to U.S. Provisional Patent Application Ser. No. 62/382,653, filed on Sep. 1, 2016, which is incorporated herein by reference.

### TECHNICAL FIELD

This disclosure relates to methods of medical treatment for preventing and treating eosinophilic gastrointestinal diseases.

### BACKGROUND

Eosinophilic Gastrointestinal Diseases (EGIDs, e.g., eosinophilic esophagitis, eosinophilic gastritis, eosinophilic gastroenteritis, eosinophilic enteritis, and eosinophilic colitis) are disorders that primarily affect the gastrointestinal (GI) tract in which eosinophil-rich inflammation occurs in the absence of known causes for eosinophilia (e.g., drug reactions, parasitic infections, and malignancy). Eosinophils are a constitutive component of the columnar-lined gastrointestinal tract and play an important role in allergic responses and parasitic infections. EGIDs are uncommon gastrointestinal diseases affecting adults and children, which have been strongly associated with food allergies, and atopic diseases or a family history of allergies. EGIDs can affect patients of any age but are more commonly seen in the third through fifth decades with a male predominance outside of the pediatric age group. EGIDs typically occur independent of peripheral blood eosinophil (>50% of the time) [4], indicating the potential significance of GI-specific mechanisms for regulating eosinophil levels. Evidence in support of the concept that EGIDs arise as a result of the interplay of genetic and environmental factors is accumulating. Notably, a large percentage (approximately 10%) of patients with EGIDs have an immediate family member with an EGID.

One EGID, eosinophilic esophagitis (EoE), is a chronic allergen-mediated clinicopathologic disease of the esophagus. A histologic and diagnostic hallmark of EoE is the accumulation of an eosinophil-predominant inflammatory infiltrate within esophageal mucosa. EoE, like other atopic diseases, is growing in incidence and prevalence (Immunotherapy 2014, 6(3):321-31). Existing medical treatments of EoE are limited, including topical corticosteroids, diet restrictions, and esophageal dilation. However, each of these approaches have been associated with some side effects and negative impact on quality of life. Moreover, the recent use of available biologics targeting eosinophil's have not achieved clinical efficacy, leading to the need for identification of new therapeutic targets.

Thus, there is a need for additional and more effective treatment and prevention methods for EGIDs, and particularly eosinophilic esophagitis.

### SUMMARY

The present inventors have discovered that reducing and/or inhibiting GM-CSF is protective in a pre-clinical model of EGIDs. They used anti-GM-CSF antibody treatments to evaluate the inflammatory patterns related to both esophageal eosinophilia and basal zone hyperplasia as a measure of post-inflammatory epithelial remodeling. Using a previously-described L2-IL5^{OXA} mouse model of EoE (Masterson J C, et al. Gut 2014; 63(1):43-53), the inventors have discovered that treatment with an anti-mouse GM-CSF monoclonal antibody significantly reduces epithelial eosinophilia as well as basal cell hyperplasia.

Granulocyte-macrophage colony-stimulating factor (GM-CSF) was first defined for its ability to generate colonies of mature granulocytes and macrophages from myeloid progenitors in vitro. GM-CSF is now known as a key mediator of eosinophilopoiesis, which is also influenced by interleukin (IL)-5 and IL-3. GM-CSF is induced by bacterial endotoxins and certain cytokines in many cell types including leukocytes (i.e., macrophage, mast cells and T cells) and non-leukocytes (i.e., fibroblasts, endothelial, mesothelial and epithelial cells) acting on cells expressing the GM-CSF-Rα which are mostly of myeloid origin, including eosinophils, basophils, DC-like cells, monocytes/macrophages, and neutrophils. Besides eosinophil progenitor proliferation and maturation, GM-CSF is now recognized to have a range of functions on mature eosinophils including, in a dose dependent manner, involvement in eosinophil priming, migration and degranulation.

This disclosure provides methods of treating eosinophilic gastrointestinal disease (EGID) by administering a human monoclonal antibody, or fragment thereof, which specifically binds to and neutralizes primate granulocyte macrophage colony stimulating factor (GM-CSF) to a subject, wherein the antibody diminishes epithelial eosinophilia and basal cell hyperplasia in the subject. These methods, the anti-GM-CSF antibody may not inhibit eosinophil maturation or activation. The anti-GM-CSF antibody may reduce the number of one or more of cells selected from mast cells, eosinophils, and basophils, in the subject. The anti-GM-CSF antibody may reduce the number of such cells from the colon, the stomach, the small intestine, the esophagus, the distal esophagus, the middle esophagus, and/or the lamina propria. In these methods, the anti-GM-CSF antibody does not reduce the number of cells of muscle tissue and/or peripheral eosinophils.

In these methods, the subject may be suffering from one or more of eosinophilic esophagitis, eosinophilic gastritis, eosinophilic gastroenteritis, eosinophilic enteritis, eosinophilic colitis, and EGID that is not adequately controlled by dietary restrictions and/or a corticosteroid. In these methods, the subject is preferably a human.

The anti-GM-CSF antibodies administered these methods may be an IgG subclass antibody, in particular an IgG1 or IgG4 subclass antibody. The anti-GM-CSF antibody may be a monoclonal antibody. The anti-GM-CSF antibody may be namilumab.

The primate GM-CSF may be a human GM-CSF.

In these methods, the antibody or fragment thereof may be administered in combination with one or more anti-inflammatory agents.

This disclosure also provides methods for treating a disorder mediated by GM-CSF expressing cells by administering an effective amount of an anti-GM-CSF antibody to a subject, wherein the antibody depletes GM-CSF expressing cells in the gastrointestinal tract of the subject.

This disclosure also provides methods for reducing the level of a cytokine in a subject by administering an effective amount of an anti-GM-CSF antibody to a subject, wherein the antibody depletes GM-CSF expressing cells in the gastrointestinal tract of the subject.

This disclosure also provides an anti-granulocyte macrophage colony stimulating factor (GM-CSF) antibody for use in the treatment of eosinophilic gastrointestinal disorders (EGIDs).

This disclosure also provides for the use of an anti-granulocyte macrophage colony stimulating factor (GM-CSF) antibody for the manufacture of a medicament for eosinophilic gastrointestinal disorders (EGIDs).

This Summary is neither intended nor should it be construed as being representative of the full extent and scope of the present disclosure. Moreover, references made herein to “the present disclosure,” or aspects thereof, should be understood to mean certain embodiments of the present disclosure and should not necessarily be construed as limiting all embodiments to a particular description. The present disclosure is set forth in various levels of detail in this Summary as well as in the attached drawings and the Description of Embodiments and no limitation as to the scope of the present disclosure is intended by either the inclusion or non-inclusion of elements, components, etc. in this Summary. Additional aspects of the present disclosure will become more readily apparent from the Description of Embodiments, particularly when taken together with the drawings.

### BRIEF DESCRIPTION OF DRAWINGS

FIGS. 1A-1C show a molecular analysis of GM-CSF expression in L2-IL5^{OXA} mouse EoE. FIG. 1A shows mRNA and FIG. 1B shows protein expression in control (L2-IL5^{VEH}/WT^{OXA}) and EoE (L2-IL5^{OXA}) mouse esophagi. FIG. 1C shows mRNA expression of GM-CSF transcript by esophageal epithelial cells following in vitro stimulation with a dose range of rhGM-CSF. Statistical significance was assessed using the Students' t-test *P<0.05. Data are expressed as means±SEM.

FIGS. 2A and 2B show that GM-CSF-Rα expression is mostly found on eosinophils in L2-IL5^{OXA} mouse EoE. Flow cytometric analysis was used to prepare histographical representations of GM-CSF-Rα expression levels on cell populations from the esophagus of L2-IL5^{OXA} EoE mice. FIG. 2A is a bar graph representation of mean fluorescence intensities (MFI's) for GM-CSF-Rα on selected cell populations of the esophagus of L2-IL5^{OXA} EoE mice. FIG. 2B shows the absolute abundance of selected leukocyte populations per esophagus of L2-IL5^{OXA} EoE mice. Holm-Sidak's correction for multiple comparisons 1-way ANOVA-vs- to control. *P<0.05, **P<0.01, ***P<0.001. An immunofluorescence examination of esophageal tissue sections from L2-IL5^{OXA} EoE mice were prepared for GM-CSFRα, SiglecF, and double positive cells.

FIGS. 3A-3D depict esophageal epithelial eosinophilia, as assessed by MBP immunohistochemistry. FIG. 3A is a schematic of the induction of mouse EoE (L2-IL5^{OXA}) and the anti-GM-CSF treatment regimen used in these studies. Anti-GM-CSF antibody: Clone MP122E9, R&D Systems MAB415. 0.5 mg/mouse per dose. (p.c. percutaneous, ie. intra-esophageal, i.p. intra-peritoneal administration.) MBP photomicrographs were formed from the distal esophagus of control anti-IgG_{2A} treated L2-IL5^{OXA} EoE mice and anti-GM-CSF treated L2-IL5^{OXA} EoE mice. Eosinophils were quantified per high-powered field in each tissue compartment (epithelial (FIG. 3B), lamina propria (FIG. 3C), muscle (FIG. 3D)) and esophageal site (proximal, middle, distal) and compared between L2-IL5^{OXA} EoE mice that underwent anti-GM-CSF to those that underwent anti-IgG_{2A} control antibody treatment. Statistical significance was assessed using the Students' t-test *P<0.05. Data are expressed as means±SEM.

FIGS. 4A and 4B demonstrate that GM-CSF treatment does not affect eosinophil activation or maturation in L2-IL5OXA mice. Flow cytometry was used to identify esophageal eosinophils following anti-GM-CSF treatment. FIG. 4A shows the analysis of maturation and activation markers on eosinophils in L2-IL5^{OXA} EoE mouse esophagi comparing anti-GM-CSF antibody treated to IgG_{2A}-isotype control antibody treated mice. FIG. 4B shows an analysis of distal whole esophageal total mRNA for levels of the transcripts for CCL11 and CCL24 in L2-IL5^{OXA} EoE mice comparing anti-GM-CSF to anti-IgG_{2A} control antibody treated groups. No statistically significant differences were observed using Students' t-test.

FIG. 5 shows an analysis of esophageal epithelial basal cell hyperplasia and proliferation by Ki67 immunohistochemistry. Ki67 photomicrographs from the distal esophagus of control anti-IgG treated L2-IL5^{OXA} EoE mice and anti-GM-CSF treated L2-IL5^{OXA} EoE mice were prepared. FIG. 5 shows the total numbers of Ki67-positive proliferating epithelial cells per high-powered field quantified and compared between L2-IL5^{OXA} EoE mice that underwent anti-GM-CSF to those that underwent anti-IgG control antibody treatment. Statistical significance was assessed using the Students' t-test *P<0.05. Data are expressed as means±SEM.

FIGS. 6A-6I show GM-CSF blockade attenuated angiogenesis and vascular remodeling in L2-IL5OXA mice. PECAM photomicrographs were prepared from the distal esophagus of anti-IgG-treated L2-IL5OXA eosinophilic esophagitis (EoE) mice and anti-GM-CSF-treated L2-IL5OXA EoE mice. FIG. 6A shows an average density of PECAM-positive vessels per mm2 was quantified and compared between L2-IL5OXA EoE mice that underwent anti-GM-CSF to those that underwent anti-IgG control antibody treatment. Analysis of distal whole esophageal total mRNA for levels of the transcripts for ANG (FIG. 6B), ANGPT1 (FIG. 6C), ANGPT2 (FIG. 6D), PECAM (FIG. 6E), vWF (FIG. 6F), CDH5 (FIG. 6G), VEGF-A (FIG. 6H), and VCAM (FIG. 6I) in L2-IL5OXA EoE mice comparing anti-GM-CSF to anti-IgG2A control antibody-treated groups. In each of these bar graphs, IgG2A isotype is shown as the open bar, and Anti-GM-CSF is shown as the solid bar. Statistical significance was assessed using the Students' t-test *P<0.05. Data are expressed as means±SEM.

### DESCRIPTION OF EMBODIMENTS

The present disclosure is drawn to methods of treating or preventing eosinophilic gastrointestinal diseases (EGIDs). Accordingly, one aspect of this disclosure relates to the administration of a human monoclonal antibody or fragment thereof which specifically binds to and neutralizes primate GM-CSF to a subject in need of such administration.

The term “specifically binds” or related expressions such as “specific binding”, “binding specifically”, “specific binder” etc. as used herein refer to the ability of the human monoclonal antibody or fragment thereof to discriminate between primate GM-CSF and any number of other potential antigens different from primate GM-CSF to such an extent that, from a pool of a plurality of different antigens as potential binding partners, only primate GM-CSF is bound, or is significantly bound. Within the meaning of this disclosure, primate GM-CSF is “significantly” bound when, from among a pool of a plurality of equally accessible different antigens as potential binding partners, primate GM-CSF is bound at least 10-fold, preferably 50-fold, most preferably 100-fold or greater more frequently (in a kinetic sense) than any other antigen different than primate GM-CSF. Such kinetic measurements can be performed on a Biacore apparatus.

As used herein, “neutralization,” “neutralizer,” “neutralizing” and grammatically related variants thereof refer to partial or complete attenuation of the biological effect(s) of GM-CSF. Such partial or complete attenuation of the biological effect(s) of GM-CSF results from modification, interruption and/or abrogation of GM-CSF-mediated signal transduction, as manifested, for example, in intracellular signaling, cellular proliferation or release of soluble substances, up- or down-regulation of intracellular gene activation, for example that resulting in expression of surface receptors for ligands other than GM-CSF. As one of skill in the art understands, there exist multiple modes of determining whether an agent, for example an antibody in question, or fragment thereof, is to be classified as a neutralizer. As an example, this may be accomplished by a standard in vitro test performed generally as follows: In a first proliferation experiment, a cell line, the degree of proliferation of which is known to depend on the activity of GM-CSF, is incubated in a series of samples with varying concentrations of GM-CSF, following which incubation the degree of proliferation of the cell line is measured. From this measurement, the concentration of GM-CSF allowing half-maximal proliferation of the cells is determined. A second proliferation experiment is then performed employing in each of a series of samples the same number of cells as used in the first proliferation experiment, the above-determined concentration of GM-CSF and, this time, varying concentrations of an antibody or fragment thereof suspected of being a neutralizer of GM-CSF. Cell proliferation is again measured to determine the concentration of antibody or fragment thereof sufficient to effect half-maximal growth inhibition. If the resulting graph of growth inhibition vs. concentration of antibody (or fragment thereof) is sigmoidal in shape, resulting in decreased cell proliferation with increasing concentration of antibody (or fragment thereof), then some degree of antibody-dependent growth inhibition has been effected, i.e. the activity of GM-CSF has been neutralized to some extent. In such a case, the antibody or fragment thereof may be considered a “neutralizer” in the sense of this disclosure. One example of a cell line, the degree of proliferation of which is known to depend on the activity of GM-CSF, is the TF-1 cell line, as described in Kitamura, T. et al. (1989). J Cell Physiol 140, 323-34.

As one of ordinary skill in the art understands, the degree of cellular proliferation is not the only parameter by which neutralizing capacity may be established. For example, measurement of the level of signaling molecules (e.g. cytokines), the level of secretion of which depends on GM-CSF, may be used to identify a suspected GM-CSF neutralizer.

Other examples of cell lines which can be used to determine whether an antibody in question or fragment thereof is a neutralizer of primate GM-CSF activity include AML-193 (Lange, B. et al. (1987). Blood 70, 192-9); GF-D8 (Rambaldi, A. et al. (1993). Blood 81, 1376-83); GM/SO (Oez, S. et al. (1990). Experimental Hematology 18, 1108-11); MOTE (Avanzi, G. C. et al. (1990). Journal of Cellular Physiology 145, 458-64); TALL-103 (Valtieri, M. et al. (1987). Journal of Immunology 138, 4042-50); UT-7 (Komatsu, N. et al. (1991). Cancer Research 51, 341-8).

This disclosure provides methods of treating a gastrointestinal disorder mediated by GM-CSF. These methods comprise administering to an individual having such disorder an effective amount of an anti-GM-CSF antibody. In one such embodiment, the method further comprises administering to the individual an effective amount of at least one additional therapeutic agent, as described below. The disorder may be an EGID. The EGID may be eosinophilic esophagitis (EOE). An “individual” according to any of the embodiments of this disclosure may be a human.

In a further aspect, this disclosure provides methods of depleting GM-CSF-expressing cells (e.g., mast cells, eosinophils, basophils) in the subject and/or reducing level of one or more cytokines, enzymes or other inflammatory mediators in a subject. In embodiments, the methods comprise administering to the subject an effective amount of an anti-GM-CSF antibody to deplete GM-CSF-expressing cells, and/or reduce one or more cytokines, in the gastrointestinal tract, esp. in the esophagus.

The anti-GM-CSF antibodies may be administered either alone or in combination with other agents in a therapy. For instance, the anti-GM-CSF antibodies may be co-administered with at least one additional therapeutic agent. In certain embodiments, an additional therapeutic agent is a corticosteroid, including an inhaled corticosteroid, a long acting muscarinic agonist, a leukotriene receptor antagonist, a mast cell inhibitor (e.g., cromolyn), a GM-CSF small molecule inhibitor, or combinations thereof.

Such combination therapies noted above encompass combined administration (where two or more therapeutic agents are included in the same or separate formulations), and separate administration, in which case, administration of the anti-GM-CSF antibody can occur prior to, simultaneously, and/or following, administration of the additional therapeutic agent.

The anti-GM-CSF antibodies (and any additional therapeutic agent) may be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein.

In these methods, anti-GM-CSF antibodies would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The antibody need not be, but is optionally formulated with one or more agents currently used to prevent or treat the disorder in question. The effective amount of such other agents depends on the amount of antibody present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of disease, the appropriate dosage of an anti-GM-CSF antibody (when used alone or in combination with one or more other additional therapeutic agents) will depend on the type of disease to be treated, the type of antibody, the severity and course of the disease, whether the antibody is administered for preventive or therapeutic purposes, previous therapy, the patient's clinical history and response to the antibody, and the discretion of the attending physician. The antibody is suitably administered to the patient at one time or over a series of treatments. Depending on the type and severity of the disease, about 1 μg/kg to 15 mg/kg (e.g. 0.1 mg/kg-10 mg/kg) of antibody can be an initial candidate dosage for administration to the patient, whether, for example, by one or more separate administrations, or by continuous infusion. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. One exemplary dosage of the antibody would be in the range from about 0.05 mg/kg to about 10 mg/kg. Thus, one or more doses of about 0.5 mg/kg, 2.0 mg/kg, 4.0 mg/kg or 10 mg/kg (or any combination thereof) may be administered to the patient. Such doses may be administered intermittently, e.g. every week or every three weeks (e.g. such that the patient receives from about two to about twenty, or e.g. about six doses of the antibody). An initial higher loading dose, followed by one or more lower doses may be administered. However, other dosage regimens may be useful. The progress of this therapy is easily monitored by conventional techniques and assays.

It is understood that any of the above formulations or therapeutic methods may be carried out using an immunoconjugate in place of, or in addition to an anti-GM-CSF antibody.

Another aspect of this disclosure provides a use of a human monoclonal anti-GM-CSF antibody or fragment thereof, as described herein, in the manufacture of a medicament, optionally comprising one or more anti-inflammatory agents, for the treatment of EGIDs. Of special interest is the use of the human monoclonal antibody or fragment thereof according to this disclosure for the preparation of a medicament for the treatment of eosinophilic esophagitis (EOE).

A further aspect of this disclosure provides a use of a human monoclonal anti-GM-CSF antibody, or fragment thereof, as described herein, optionally comprising one or more additional anti-inflammatory agents, for the treatment of EGIDs. Of special interest is the use of the human monoclonal antibody or fragment thereof according to this disclosure for the treatment of eosinophilic esophagitis (EOE).

The human antibody or fragment thereof of the methods of this disclosure may be monoclonal. As used herein, the term “monoclonal” is to be understood as having the meaning typically ascribed to it in the art, namely an antibody (or its corresponding fragment) arising from a single clone of an antibody-producing cell such as a B cell, and recognizing a single epitope on the antigen bound. It is particularly difficult to prepare human antibodies which are monoclonal. In contrast to fusions of murine B cells with immortalized cell lines, fusions of human B cells with immortalized cell lines are not viable. Thus, a human monoclonal antibody is the result of overcoming significant technical hurdles generally acknowledged to exist in the field of antibody technology. The monoclonal nature of an antibody makes it particularly well suited for use as a therapeutic agent, since such antibody will exist as a single, homogeneous molecular species which can be well-characterized and reproducibly made and purified. These factors result in a product whose biological activity can be predicted with a high level of precision, very important if such a molecule is going to gain regulatory approval for therapeutic administration in humans.

It is preferable that the monoclonal antibody (or corresponding fragment) be a human antibody (or corresponding fragment). In contemplating an antibody agent intended for therapeutic administration to humans, it is highly advantageous that this antibody is of human origin. Following administration to a human patient, a human antibody or fragment thereof will most probably not elicit a strong immunogenic response by the patient's immune system, i.e. will not be recognized as being a “foreign”, that is non-human protein. This means that no host, i.e. patient antibodies will be generated against the therapeutic antibody which would otherwise block the therapeutic antibody's activity and/or accelerate the therapeutic antibody's elimination from the body of the patient, thus preventing it from exerting its desired therapeutic effect.

The term “human” antibody as used herein is to be understood as meaning that the antibody, or its fragment, comprises (an) amino acid sequence(s) contained in the human germline antibody repertoire. For the purposes of definition herein, an antibody, or its fragment, may therefore be considered human if it consists of such (a) human germline amino acid sequence(s), i.e. if the amino acid sequence(s) of the antibody in question or fragment thereof is (are) identical to (an) expressed human germline amino acid sequence(s). An antibody or fragment thereof may also be regarded as human if it consists of (a) sequence(s) that deviate(s) from its (their) closest human germline sequence(s) by no more than would be expected due to the imprint of somatic hypermutation. Additionally, the antibodies of many non-human mammals, for example rodents such as mice and rats, comprise VH CDR3 amino acid sequences which one may expect to exist in the expressed human antibody repertoire as well. Any such sequence(s) of human or non-human origin which may be expected to exist in the expressed human repertoire would also be considered “human” for the purposes of this disclosure.

These human monoclonal antibodies, or fragments thereof, may exhibit cross reactivity between both human and at least one monkey species. This is especially advantageous for an antibody molecule which is intended for therapeutic administration in human subjects, since such an antibody will normally have to proceed through a multitude of tests prior to regulatory approval, of which certain early tests involve non-human animal species. The human monoclonal antibody may be an IgG antibody. As is well known in the art, an IgG comprises not only the variable antibody regions responsible for the highly discriminative antigen recognition and binding, but also the constant regions of the heavy and light antibody polypeptide chains normally present in endogenously produced antibodies and, in some cases, even decoration at one or more sites with carbohydrates. Such glycosylation is generally a hallmark of the IgG format, and portions of these constant regions make up the Fc region of a full antibody which is known to elicit various effector functions in vivo. In addition, the Fc region mediates binding of IgG to Fc receptor, hence prolonging half-life in vivo as well as facilitating homing of the IgG to locations with increased Fc receptor presence, e.g. inflamed tissue. Advantageously, the IgG antibody is an IgG1 antibody or an IgG4 antibody, formats which are preferred since their mechanism of action in vivo is particularly well understood and characterized. This is especially the case for IgG1 antibodies.

Fragments of these human monoclonal antibodies that may function in the methods of this disclosure may include an scFv, a single domain antibody, an Fv, a VHH antibody, a diabody, a tandem diabody, a Fab, a Fab′ or a F(ab)2. These formats may generally be divided into two subclasses, namely those which consist of a single polypeptide chain, and those which comprise at least two polypeptide chains. Members of the former subclass include an scFv (comprising one VH region and one VL region joined into a single polypeptide chain via a polypeptide linker); a single domain antibody (comprising a single antibody variable region) such as a VHH antibody (comprising a single VH region). Members of the latter subclass include an Fv (comprising one VH region and one VL region as separate polypeptide chains which are non-covalently associated with one another); a diabody (comprising two non-covalently associated polypeptide chains, each of which comprises two antibody variable regions—normally one VH and one VL per polypeptide chain—the two polypeptide chains being arranged in a head-to-tail conformation so that a bivalent antibody molecule results); a tandem diabody (bispecific single-chain Fv antibodies comprising four covalently linked immunoglobulin variable-VH and VL-regions of two different specificities, forming a homodimer that is twice as large as the diabody described above); a Fab (comprising as one polypeptide chain an entire antibody light chain, itself comprising a VL region and the entire light chain constant region and, as another polypeptide chain, a part of an antibody heavy chain comprising a complete VH region and part of the heavy chain constant region, said two polypeptide chains being inter-molecularly connected via an interchain disulfide bond); a Fab′ (as a Fab, above, except with additional reduced disulfide bonds comprised on the antibody heavy chain); and a F(ab)2 (comprising two Fab′ molecules, each Fab′ molecule being linked to the respective other Fab′ molecule via interchain disulfide bonds). In general, antibody fragments of the type described hereinabove allow great flexibility in tailoring, for example, the pharmacokinetic properties of an antibody desired for therapeutic administration to the particular exigencies at hand. For example, it may be desirable to reduce the size of the antibody administered in order to increase the degree of tissue penetration when treating tissues known to be poorly vascularized (for example, joints). Under some circumstances, it may also be desirable to increase the rate at which the therapeutic antibody is eliminated from the body, said rate generally being accelerated by decreasing the size of the antibody administered.

According to a further embodiment, said human monoclonal antibody or fragment thereof may be present in monovalent monospecific; multivalent monospecific, in particular bivalent monospecific; or multivalent multispecific, in particular bivalent bispecific forms. In general, a multivalent monospecific, in particular bivalent monospecific antibody such as a full human IgG as described hereinabove may bring with it the therapeutic advantage that the neutralization effected by such an antibody is potentiated by avidity effects, i.e. binding by the same antibody to multiple molecules of the same antigen, here primate GM-CSF. Several monovalent monospecific forms of fragments of antibodies have been described above (for example, an scFv, an Fv, a VHH or a single domain antibody). Multivalent multispecific, in particular bivalent bispecific forms of the human monoclonal anti-primate GM-CSF antibody may include a full IgG in which one binding arm binds to primate GM-CSF while the other binding arm of which binds to another antigen different from primate GM-CSF. A further multivalent multispecific, in particular bivalent bispecific form may advantageously be a human single chain bispecific antibody, i.e. a recombinant human antibody construct comprising two scFv entities as described above, connected into one contiguous polypeptide chain by a short, interposed polypeptide spacer as generally known in the art (see for example WO 99/54440 for an anti-CD19×anti-CD3 bispecific single chain antibody). Here, one scFv portion of the bispecific single chain antibody comprised within the bispecific single chain antibody will specifically bind primate GM-CSF as set out above, while the respective other scFv portion of this bispecific single chain antibody will bind another antigen determined to be of therapeutic benefit.

According to a further embodiment the human monoclonal antibody or fragment thereof may be derivatized, for example with an organic polymer, for example with one or more molecules of polyethylene glycol (“PEG”) and/or polyvinyl pyrrolidone (“PVP”). As is known in the art, such derivatization can be advantageous in modulating the pharmacodynamic properties of antibodies or fragments thereof. Especially preferred are PEG molecules derivatized as PEG-maleimide, enabling conjugation with the antibody or fragment thereof in a site-specific manner via the sulfhydryl group of a cysteine amino acid. Of these, especially preferred are 20 kD and/or 40 kD PEG-maleimide, in either branched or straight-chain form. It may be especially advantageous to increase the effective molecular weight of smaller human anti-primate GM-CSF antibody fragments such as scFv fragments by coupling the latter to one or more molecules of PEG, especially PEG-maleimide.

An anti-GM-CSF antibody useful in the methods of this disclosure may be namilumab, a neutralizing human IgG1 anti-GM-CSF monoclonal antibody developed by Amgen (Thousand Oaks, Calif.).

In the methods of this disclosure, the anti-GM-CSF antibody or fragment thereof may be administered as a pharmaceutical composition comprising a human monoclonal antibody or fragment thereof. In accordance with this disclosure, the term “pharmaceutical composition” relates to a composition for administration to a patient, preferably a human patient. In a preferred embodiment, the pharmaceutical composition comprises a composition for parenteral, transdermal, intraluminal, intraarterial, intrathecal and/or intranasal administration or by direct injection into tissue. It is in particular envisaged that said pharmaceutical composition is administered to a patient via infusion or injection. Administration of the suitable compositions may be effected by different ways, e.g., by intravenous, intraperitoneal, subcutaneous, intramuscular, topical or intradermal administration. The pharmaceutical compositions may further comprise a pharmaceutically acceptable carrier. Examples of suitable pharmaceutical carriers are well known in the art and include phosphate buffered saline solutions, water, emulsions, such as oil/water emulsions, various types of wetting agents, sterile solutions, liposomes, etc. Compositions comprising such carriers can be formulated by conventional methods. These pharmaceutical compositions can be administered to the subject at a suitable dose. The dosage regimen will be determined by the attending physician and clinical factors. As is well known in the medical arts, dosages for any one patient depend upon many factors, including the patient's size, body surface area, age, the particular compound to be administered, sex, time and route of administration, general health, and other drugs being administered concurrently. Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, inert gases and the like. In addition, the pharmaceutical compositions may comprise proteinaceous carriers, like, e.g., serum albumin or immunoglobulin, preferably of human origin. It is envisaged that the pharmaceutical composition may comprise, in addition to the human monoclonal antibody or fragment thereof, further biologically active agents, depending on the intended use of the pharmaceutical composition. Such agents might be drugs acting on the gastro-intestinal system, drugs acting as cytostatica, drugs preventing hyperurikemia, drugs inhibiting immunoreactions (e.g. corticosteroids), drugs modulating the inflammatory response, drugs acting on the circulatory system and/or agents such as cytokines known in the art.

Each publication or patent cited herein is incorporated herein by reference in its entirety.

The disclosure now being generally described will be more readily understood by reference to the following examples, which are included merely for the purposes of illustration of certain aspects of the embodiments of the present disclosure. The examples are not intended to limit the disclosure, as one of skill in the art would recognize from the above teachings and the following examples that other techniques and methods can satisfy the claims and can be employed without departing from the scope of the claimed disclosure.

### EXAMPLES

Materials and Methods Used in these Examples

### Mice:

All studies were performed with male or female L2-IL5 mice on a C57BL/6J background generated as previously described (Masterson J C, et al. Gut 2014; 63(1):43-53. Animals were maintained in micro-isolator cages housed in a specific pathogen-free (SPF) facility at the University of Colorado. Age and sex-matched L2-IL5 mice were used as controls. Studies involving animals were performed in accordance with National Institutes of Health, University of Colorado IACUC guidelines.

### Induction of Experimental EoE in L2-IL5^{OXA} Mice Using 4-Ethoxymethylene-2-Phenyl-2-Oxazolin-5-One (Oxazolone or OXA):

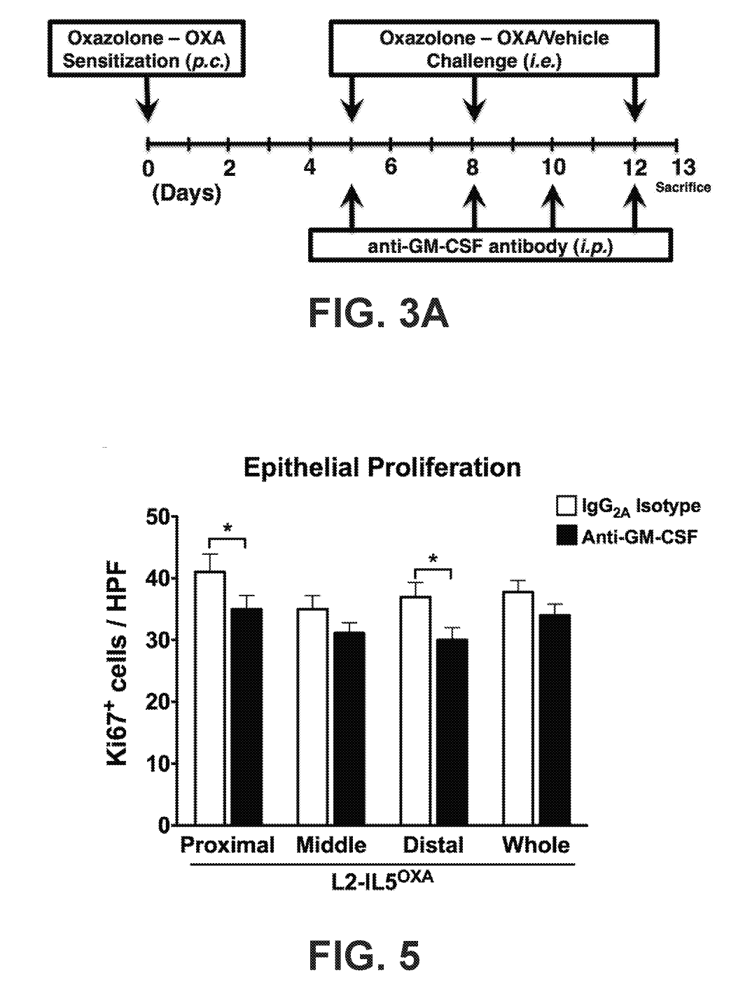

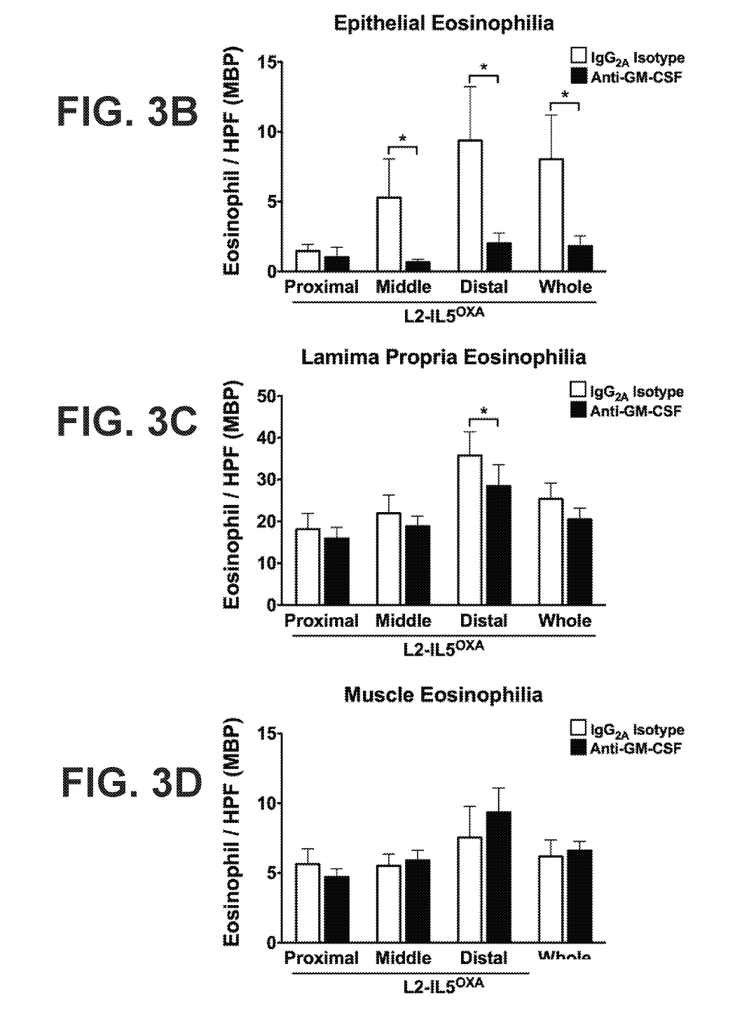

Induction of esophageal eosinophilic inflammation in mice (L2-IL5^{OXA}EoE) was established using a 4-Ethoxymethylene-2-phenyl-2-oxazolin-5-one (oxazolone or OXA) (Sigma, St Louis, Mo.) contact hypersensitivity protocol as previously described (FIG. 3A; and Masterson J C, et al., Gut 2014, supra). Briefly, on day 0 of the protocol, anesthetized mice were shaved on the abdomen and oxazolone was applied to the skin surface (150 μl of a 3% (w/v) solution of OXA in 4:1 acetone-olive oil vehicle) to initiate the sensitization phase of the protocol. On days 5, 8 and 12 mice were challenged by an intra-esophageal gavage of 100 μl of a 1% (w/v) OXA in 30% ethanol/olive oil vehicle. Vehicle control L2-IL5 mice were sensitized as above and challenged with vehicle alone. All mice were assessed 24 hours following the last OXA challenge (protocol day 13).

In some studies, a GM-CSF depleting antibody was administered to mice. These studies were completed using a monoclonal rat IgG2a antibody specific for GM-CSF (Clone # MP122E9) or monoclonal rat IgG2a isotype control antibody (Clone #54447) (R&D Systems, Minneapolis, Minn., USA). Experimental animals were injected with four intraperitoneal (i.p.) doses of anti-GM-CSF antibody (0.5 mg/mouse) or IgG2a isotype control on days 5, 8, 10 and 12 during experimental esophagitis. Mice were sacrificed 24 hours following final treatment (day 13).

### Tissue Processing and Immunohistochemical Assessment of Tissues:

Whole length esophageal tissues were removed and fixed with 10% neutral buffered formalin, processed, paraffin embedded and cut into 5 μm sections. Sections were stained with hematoxylin and eosin (H&E) (Sigma, St Louis, Mo., USA) or subjected to immunohistochemistry for eosinophil major basic protein-1 (MBP-1; Clone MT-14.7) (Lee Labs, Mayo Clinic, AZ, USA) or Ki67 (Dako, Carpinteria, Calif., USA) to assess cell proliferation, both as previously described (Masterson J C, et al., Gut 2014; supra). MBP-1 immuno-positive cells were visualized with permanent red chemotrope and Ki67 immuno-positive cells were visualized with DAB chemotrope (Dako, Carpinteria, Calif., USA) and the slides were counterstained with Methyl Green (Vector Labs, Burlingame, Calif., USA). Control sections replaced the primary antibody in each case with a rat IgG isotype control antibody (Vector Labs, Burlingame, Calif., USA). Quantification of either MBP-1 or Ki67 immuno-positive cells was determined by gathering the numerical averages of nine non-overlapping high-power fields (0.26 mm²) per esophagus (3-distal, 3-mid and 3-proximal). Cell numbers are presented as a mean±SEM.

### Esophageal Leukocyte Isolation, Quantification and Flow Cytometric Analysis:

Esophageal leukocytes were isolated as previously described (Masterson J C, et al., Gut 2014, supra). Briefly, esophagi were resected, cut longitudinally, washed in PBS and then digested with collagenase (Sigma, St Louis, Mo., USA) as previously described for other intestinal tissues (Masterson J C, et al., Am J Pathol 2011; 179(5):2302-14). Cell counts and viability of recovered leukocytes were determined with a hemocytometer and trypan blue exclusion.

Single-cell suspensions were blocked using 1 μg/μl of Fc blocker (CD16/32; eBiosciences, San Diego, Calif., USA). Cells were then stained for 2 hours at 4 degrees with cell type-specific antibodies. Antibodies used for the staining of specific cell surface markers include GM-CSF-Rα (698423), Ly6G (1A8), Ly6C (AL-21), IL-5Rα (T21), CCR3 (83103), SiglecF (E50-2440) all (BD-Biosciences, San Jose, Calif., USA); NK1.1 (PK136), MHC-II (M5/114.15.2), FcεR1α (MAR-1), CD200R3 (Ba13), CD80 (16-10A1) all (Biolegend, San Diego, Calif., USA) and CD11b (M1/70), CD34 (RAM34), CD45 (30-f11), CD49b (DX5), CD3e (145-2C11), CD19 (1D3), EPCAM (G8.8), CD69 (H1.2F3) all (eBiosciences, San Diego, Calif., USA). Viable cells were determined with the use of Live/Dead AquaVi staining (Invitrogen, Grand Island, N.Y., USA). Flow cytometric analysis was performed using a BD FACSCanto™ II (BD Biosciences, San Jose, Calif., USA). Data files were further analyzed using FLOWJo software (Tree Star Inc, Ashland, Oreg., USA). Cell culture: EPC2-hTERT immortalized human esophageal epithelial cells were cultured as previously described (Harada H, et al., Mol Cancer Res 2003; 1(10):729-38). EPC2-hTERT cells were seeded at 60,000 cells per well of a 24-well plate and 24 hours after plating cells were switched to high calcium (1.8 mM) medium for a further 48 hours. Cells were then washed and treated for 24 hours with varying concentrations of rhGM-CSF (R&D Systems, Minneapolis, Minn., USA). Cells were harvested for mRNA analysis using RLT buffer from Qiagen RNeasy kits (Qiagen, Valencia, Calif., USA).

### RNA Isolation and Real-Time RT-PCR:

Total RNA was prepared from whole distal esophageal tissues with RNeasy Mini Kits (Qiagen, Valencia, Calif., USA) and hand-held laboratory homogenizer (PRO Scientific, Oxford, Conn., USA). First strand cDNA synthesis was performed from 500 ng of total RNA using the High Capacity cDNA archive kit (Applied BioSystems, Foster City, Calif., USA). Transcript expression was assessed using Taqman Gene Expression Assays Taqman probes (Applied Biosystems, Foster City, Calif., USA). rtRT-PCR reactions were performed with ABsolute™ Blue QPCR ROX Mix (Thermo Scientific, Surrey, UK). Thermocycling and analysis was performed with ABI-7300 System and software. Data was normalized to 18S expression and calculated as RQ (Relative Quantity; 2^{-ΔΔCt,} where Ct is cycle threshold) for each sample.

### GM-CSF Protein Assessment of Esophageal Tissue:

Esophagi were resected, cut longitudinally to expose the luminal surface, washed in PBS, snap frozen and stored at −80 C prior to use. Tissues were homogenized in MSD lysis buffer containing Roche complete mini protease inhibitor cocktail (Sigma, St Louis, Mo., USA) and assessed for GM-CSF protein content using mouse GM-CSF specific Mesoscale Assay (as per the manufacturer's instructions) (Meso Scale Diagnostics (MSD), Rockville, Md., USA) or total protein content using BCA Assay (ThermoFisher, Waltham, Mass.).

### Statistical Analysis:

Statistical analyses of data outcomes were performed by Student's t-test. Data are expressed as means±SEM. A P-value of 50.05 was considered as statistical significance although in some cases higher levels of significance are noted and described in the figure legends where applicable. *P≤0.05, **P≤0.01, ***P≤0.001.

### Example 1: GM-CSF Expression is Increased in Esophagitis

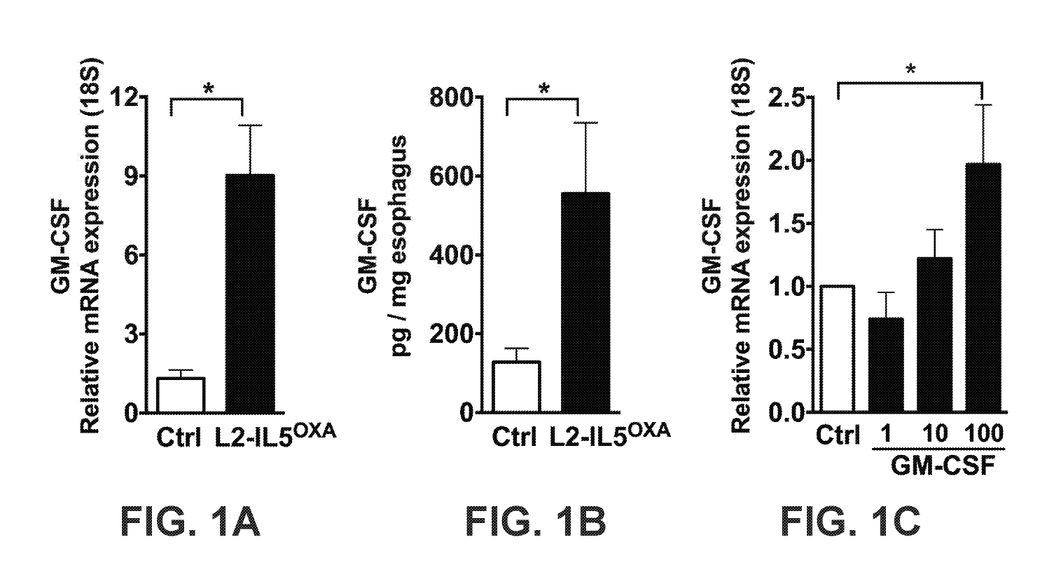

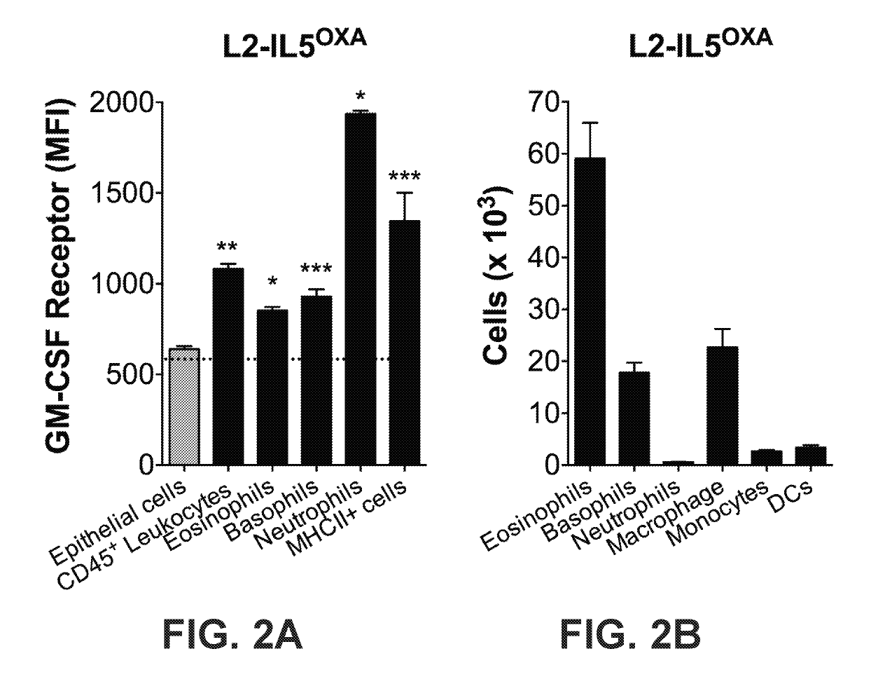

In order to understand the potential impact of GM-CSF on esophageal eosinophilia, the inventors performed 3 sets of experiments:
1) The inventors first tested to see if GM-CSF expression was elevated in the esophagus linked with the eosinophil predominant inflammation occurring in the L2-IL5^{OXA} mouse model of Eosinophilic Esophagitis (EoE). After 8 days, the inventors examined both mRNA and protein expression in the esophageal tissue and found both to be significantly increased (mRNA: 1.3±0.3 versus 9.0±1.9, P≤0.05; Protein: 128±35 versus 556±179 pg/ml, P≤0.05) (FIG. 1A-B).
2) The inventors tested whether GM-CSF would stimulate esophageal epithelial cells to release pro-allergic molecules given that epithelial cells exposed to GM-CSF are associated with perpetuation of atopic inflammatory conditions such as atopic dermatitis, (12, 13). Our results showed that exposure of the esophageal epithelium to rhGM-CSF induced the concentration-dependent production of GM-CSF in vitro (1.97±0.47-fold increase, P≤0.05) (FIG. 1C).
3) The inventors went on to examine expression of the GM-CSF-Rα in various myeloid-type leukocyte populations present during active inflammation in L2-IL5^{OXA} mouse EoE. Here the inventors found by flow cytometry that GM-CSF-Rα was expressed at highest per cell concentration on neutrophils and MHCII+ cells, and to a lesser degree on eosinophils and basophils (FIGS. 2A and 2B). Assessment of cell frequency in mouse esophagi found that neutrophils were rare compared to other leukocytes such as eosinophils (FIG. 2B). Immunofluorescent assessment of GM-CSF-Rα and SiglecF revealed co-staining for both to a significant degree compared to cells that were positive for GM-CSF-Rα alone.

### Example 2: Anti-GM-CSF Antibody Treatment Attenuates Esophageal Epithelial Eosinophilia

The inventors tested the hypothesis that depletion of GM-CSF protein by antibodies would reduce the esophageal eosinophilia in L2-IL5^{OXA} EoE mice. Experimental groups of animals were given anti-GM-CSF antibody (via intraperitoneal injection, control animals received isotype matched serum immunoglobulin) beginning day 5, the day of the first intra-esophageal challenge and on days 8, 10 and 12 after induction of esophagitis (FIG. 3A). Anti-GM-CSF treatment lead to a significant decrease in epithelial eosinophilia as measured by counting intact eosinophils and assessing eosinophil MBP-1 staining compared to anti-IgG2a isotype control treated L2-IL5^{OXA} EoE mice (FIG. 3B). The pattern of inhibition appeared to be most notable in the distal and middle esophagus. In addition, eosinophilia in the lamina propria was also reduced significantly (FIG. 3C) but no effect was observed on eosinophilia in the muscle layers (FIG. 3D).

In order to elucidate whether these effects were locally restricted within the esophagus or were as a result of effects on eosinophil development and circulation, peripheral eosinophils were assessed from the spleen and bone marrow compartments by flow cytometry (Live, SSC-hi, CD45⁺, Ly6G⁻, SiglecF⁺). No significant change was detected when quantifying peripheral eosinophilia (Bone Marrow: 3×10\hat{ } 6 vs 2.6×10\hat{ }6, P=0.46. Spleen: 13.7×10\hat{ }6 vs 12.7×10\hat{ }6, P=0.69. Anti-GM-CSF-L2-IL50^{OXA} versus IgG-Ctrl-L2-IL50^{OXA}).

### Example 3: Anti-GM-CSF Antibody Treatment does not Affect Esophageal Eosinophil Maturation, Activation Markers or Eosinophil Chemoattractant Expression

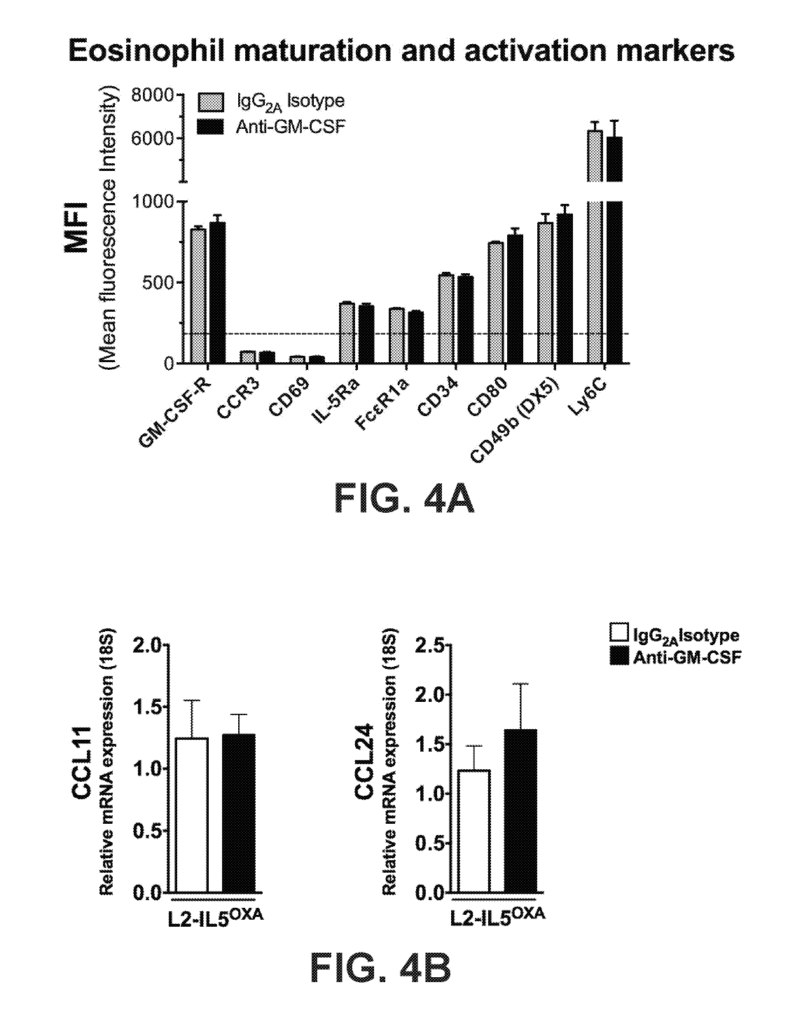

Given its role as an eosinophilopoietin the inventors examined whether depletion of GM-CSF during esophagitis would affect the maturation status of eosinophils in mice treated with anti-GM-CSF when compared to those treated with anti-IgG2a control (FIGS. 4A and 4B). The inventors identified esophageal eosinophils by flow cytometry as FSC^{hi}, SSC^{hi}, live, single cells, CD45⁺, SiglecF⁺. The inventors then examined these cells for the level of expression of various maturation and activation markers. The inventors found no effect of anti-GM-CSF on the expression levels of any of these selected markers (FIG. 4A). The inventors also examined whether anti-GM-CSF would affect esophageal tissue expression of eosinophil chemokines CCL11 and CCL24 (Eotaxins 1 & 2) and found no effect (FIG. 4B).

### Example 4: Anti-GM-CSF Antibody Treatment Resolves Basal Cell Hyperplasia and Epithelial Remodeling

The effects of anti-GM-CSF depletion on epithelial remodeling and basal cell hyperplasia were assessed. A substantial improvement in basal cell hyperplasia and the homeostatic localization of proliferative cells as demarcated by Ki67 immunohistochemistry was observed following treatment. When enumerated, the most significant improvement was observed in the proximal and distal esophagus (FIG. 5).

### Example 5: Anti-GM-CSF Antibody Treatment Decreases Blood Vessel Density and Angiogenic Factor Production

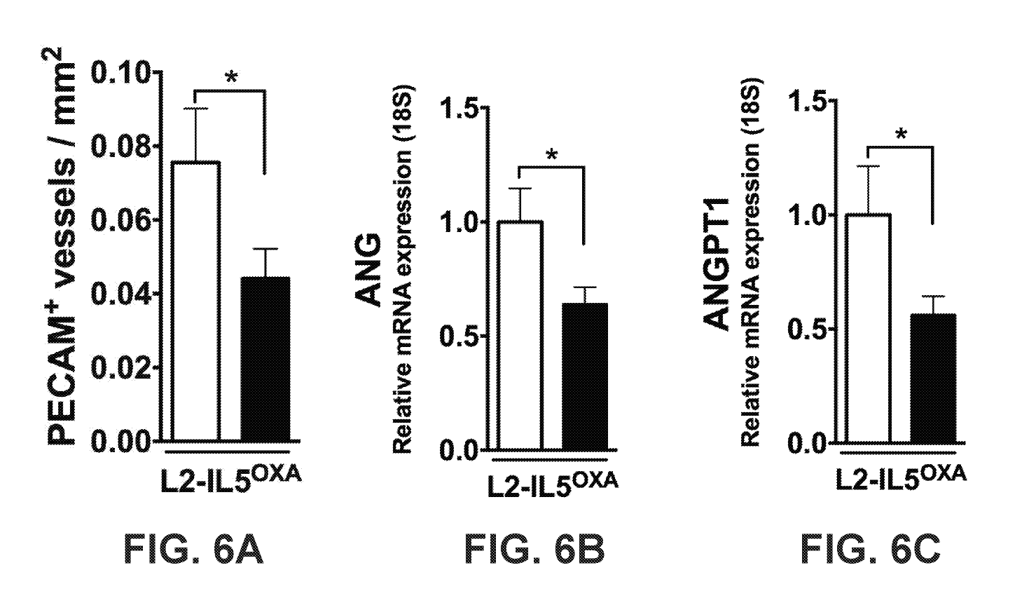

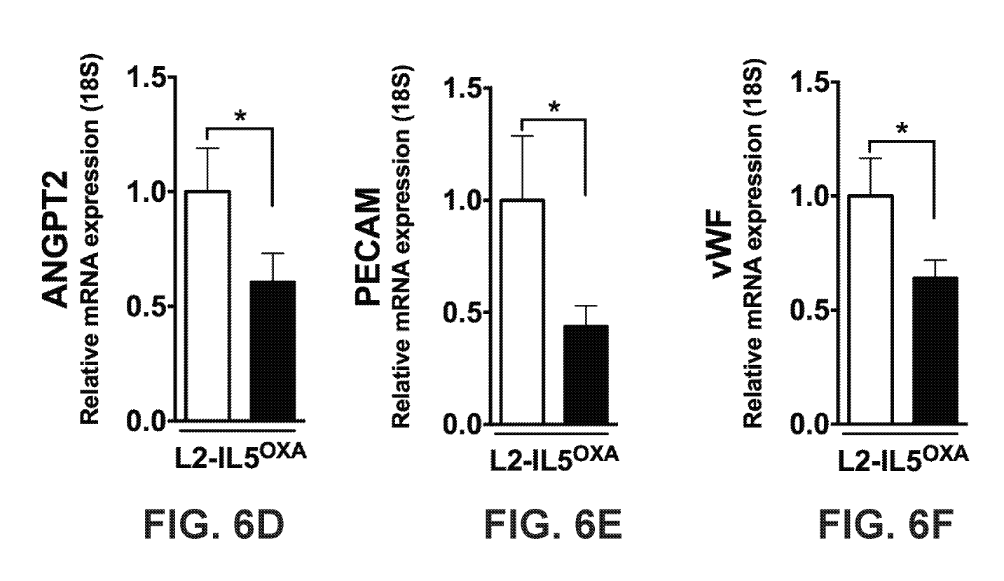

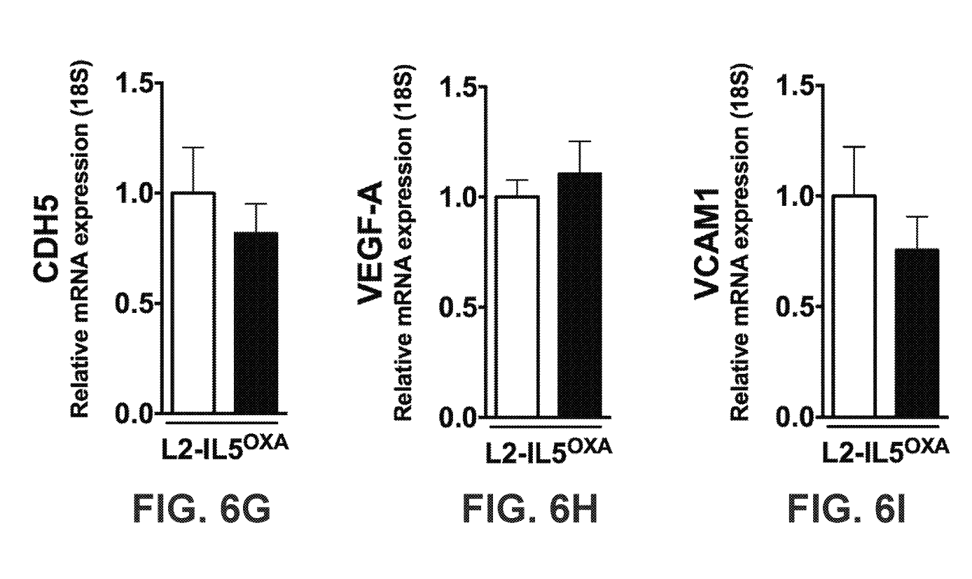

Finally, the role of GM-CSF in esophageal vascular remodeling in the context of eosinophilic inflammation was examined. PECAM (CD31)-positive vessel density was enumerated, and it was found that anti-GM-CSF treatment resulted in a lower vessel density compared to IgG control-treated L2-IL5OXA animals (FIG. 6A). When the expression of angiogenic stimulating factors including angiogenin and angiopoietins 1 and 2 was assessed, it was also determined that anti-GM-CSF treatment led to significantly reduced levels of these growth factors (FIGS. 6B-6D). In addition, GMCSF blockade significantly decreased esophageal levels of endothelial markers PECAM and von Wilebrand Factor (vWF) compared to IgG-treated controls (FIGS. 6E and 6F). Interestingly, no difference in the expression levels of the activation markers CDH5, VCAM, or VEGF-A was detected (FIGS. 6G-6I). The inventors also assessed each of these molecular markers in the context of uninflamed L2-IL5VEH esophagi and found a significant decrease in selected markers (VWF: 40% decrease ±4%, P<0.05, ANG2: 60% decrease ±11%, P<0.05, PECAM: 62% decrease ±16%, P<0.05) in the esophagi treated with anti-GM-CSF antibody when compared to L2-IL5VEH IgG control-treated animals. However, importantly, this did not lead to a decrease in the number of PECAM+ blood vessels in anti-GM-CSF antibody-treated uninflamed L2-IL5VEH mice (0.08±0.03 vs 0.07±0.02, P=0.75; anti-GM-CSF-L2-IL5VEH vs IgG-Ctrl-L2-IL5VEH). Thus, therapeutic intervention with anti-GM-CSF limits epithelial remodeling and angiogenic activation during active disease in the L2-IL5OXA model of EoE.

In these studies, the inventors directly examined the physiological impact of targeting GM-CSF, a crucial eosinophilopoietin in the pathogenesis of esophageal eosinophilia and epithelial remodeling in the L2-IL5^{OXA} mouse model of Eosinophilic Esophagitis (EoE), and demonstrated that GM-CSF expression is increased in the L2-IL5^{OXA} mouse model of EGIDs. The inventors demonstrated that targeted GM-CSF depletion results in a significant decrease in epithelial eosinophilia, as well as decreased epithelial basal cell hyperplasia resulting in overall attenuation of the development of EoE-like disease in this delayed type hypersensitivity model. Collectively, these studies demonstrate that reducing and/or inhibiting GM-CSF is effective in reducing eosinophil numbers in a mouse model of EoE.

Despite the fact that clinical intervention studies have shown that inhibition of another eosinophilopoietin, interleukin (IL)-5, significantly reduced eosinophilia these studies failed to demonstrate clinical benefit. The inventors therefore sought to understand the potential role of GM-CSF to address the need for alternative therapeutic options. Previous studies have demonstrated that targeting GM-CSF influences leukemogenesis in an in vitro model of chronic myelomonocytic leukemia, inflammatory responses in experimental arthritis, reduced tissue macrophage in rat model of myocardial infarction, reduced Amyloid beta1-42 and microglial activity in a mouse model of Alzheimer's disease. Two studies have directly examined the influence of anti-GM-CSF therapy on bronchioalveolar lavage fluid (BALf) eosinophilia in mouse models of allergic airway inflammation, and an ongoing trial is examining anti-GM-CSF antibodies role in subjects with asthma. No previous studies have been completed determining the impact of anti-GM-CSF in esophagitis.

The inventors have now shown the increased presence of GM-CSF and its role in esophageal epithelial eosinophilia using the L2-IL5^{OXA} mouse model of EoE. Previous studies using eotaxin^{-/-} genetically targeted mice in a mouse model of EoE still accumulated esophageal eosinophils, implicating additional chemotactic axes in esophageal epithelial recruitment. The inventors show that reduced epithelial eosinophilia is not as a result of effects of anti-GM-CSF treatment on esophageal eosinophil chemokines eotaxin-1 or -2, nor on eosinophil development and circulation. GM-CSF acts on mature eosinophils to elicit priming, migration and degranulation. Here the inventors show no effect of anti-GM-CSF therapy on esophageal eosinophil expression of maturation or activation markers. These findings suggest that decreased esophageal epithelial eosinophilia in the L2-IL5^{OXA} EoE mouse model may be acting through local effects of GM-CSF within the esophagus, possibly effects on intra-epithelial eosinophil survival or indirectly via other cells including antigen presenting cells. The inventors confirm the expression of esophageal GM-CSF-receptor-alpha in the L2-IL5^{OXA} mouse EoE model is primarily on eosinophils, and to a lesser extent on basophils and MHCII positive cells.

Previous studies in mouse models as well as clinical trials in EoE have targeted factors associated with eosinophilopoiesis. GM-CSF along with IL-5 and IL-3 are a triad of important, though redundant, immunological factors associated with eosinophil development in the bone marrow. The inventors' current studies show that anti-GM-CSF attenuates esophageal epithelial eosinophilia to some extent. Although attenuated EoE-like disease develops in this model despite anti-GM-CSF treatment, these studies still implicate GM-CSF in the esophageal inflammatory microenvironment influencing eosinophilia.

The foregoing examples of the present invention have been presented for purposes of illustration and description. Furthermore, these examples are not intended to limit the invention to the form disclosed herein. Consequently, variations and modifications commensurate with the teachings of the description of the invention, and the skill or knowledge of the relevant art, are within the scope of the present invention. The specific embodiments described in the examples provided herein are intended to further explain the best mode known for practicing the invention and to enable others skilled in the art to utilize the invention in such, or other, embodiments and with various modifications required by the particular applications or uses of the present invention. The appended claims should be construed to include alternative embodiments to the extent permitted by the prior art.

To the extent that the appended claims have been drafted without multiple dependencies, this has been done only to accommodate formal requirements in jurisdictions which do not allow such multiple dependencies. It should be noted that all possible combinations of features which would be implied by rendering the claims multiply dependent are explicitly envisaged and should be considered part of the invention.

1. A method of treating eosinophilic gastrointestinal disease (EGID) comprising administering a human monoclonal antibody or fragment thereof which specifically binds to and neutralizes primate granulocyte macrophage colony stimulating factor (GM-CSF) to a subject, wherein the antibody diminishes epithelial eosinophilia and basal cell hyperplasia in the subject.

2. The method of claim 1, wherein the anti-GM-CSF antibody does not inhibit eosinophil maturation or activation.

3. The method of claim 1, wherein the anti-GM-CSF antibody reduces the number of one or more of cells selected from mast cells, eosinophils, and basophils, in the subject.

4. The method of claim 3, wherein the anti-GM-CSF antibody reduces the number of cells from the colon.

5. The method of claim 3, wherein the anti-GM-CSF antibody reduces the number of cells from the stomach.

6. The method of claim 3, wherein the anti-GM-CSF antibody reduces the number of cells from the small intestine.

7. The method of claim 3, wherein the anti-GM-CSF antibody reduces the number of cells from the esophagus.

8. The method of claim 7, wherein the anti-GM-CSF antibody reduces the number of cells from the distal esophagus.

9. The method of claim 7, wherein the anti-GM-CSF antibody reduces the number of cells from the middle esophagus.

10. The method of claim 7, wherein the anti-GM-CSF antibody reduces the number of cells from the lamina propria.

11. The method of claim 3, wherein the anti-GM-CSF antibody does not reduce the number of cells of muscle tissue.

12. The method of claim 3, wherein the anti-GM-CSF antibody does not reduce the number of peripheral eosinophils.

13. The method of claim 1, wherein the subject is suffering from eosinophilic esophagitis.

14. The method of claim 1, wherein the subject is suffering from eosinophilic gastritis.

15. The method of claim 1, wherein the subject is suffering from eosinophilic gastroenteritis.

16. The method of claim 1, wherein the subject is suffering from eosinophilic enteritis.

17. The method of claim 1, wherein the subject is suffering from eosinophilic colitis.

18. The method of claim 1, wherein the subject is suffering from EGID that is not adequately controlled by dietary restrictions and/or a corticosteroid.

19. The method of claim 1, wherein the subject is a human.

20. The method of claim 1, wherein the anti-GM-CSF antibody is an IgG antibody.

21. The method of claim 20, wherein the IgG is an IgG1 or IgG4 isotype.

22. The method of claim 1, wherein the primate GM-CSF is a human GM-CSF.

23. The method of claim 1, wherein the antibody or fragment thereof is administered in combination with one or more anti-inflammatory agents.

24. The method of claim 1, wherein the anti-GM-CSF antibody is a monoclonal antibody.

25. The method of claim 1, wherein the anti-GM-CSF antibody is namilumab.

26. A method for treating a disorder mediated by GM-CSF expressing cells comprising administering an effective amount of an anti-GM-CSF antibody to a subject, wherein the antibody depletes GM-CSF expressing cells in the gastrointestinal tract of the subject.

27. A method for reducing the level of a cytokine in a subject comprising administering an effective amount of an anti-GM-CSF antibody to a subject, wherein the antibody depletes GM-CSF expressing cells in the gastrointestinal tract of the subject.

28. An anti-granulocyte macrophage colony stimulating factor (GM-CSF) antibody for use in the treatment of eosinophilic gastrointestinal disorders (EGIDs).

29. Use of an anti-granulocyte macrophage colony stimulating factor (GM-CSF) antibody for the manufacture of a medicament for eosinophilic gastrointestinal disorders (EGIDs).
